(19) United States
(12) Patent Application Publication
(10) Pub. No.: US 2024/0050605 A1
(43) Pub. Date: Feb. 15, 2024

(54) DISINFECTANT SYSTEM

(71) Applicant: H7 Technologies, Orange, CA (US)

(72) Inventor: Sherylinn Hoang, Orange, CA (US)

(21) Appl. No.: 18/230,953

(22) Filed: Aug. 7, 2023

Related U.S. Application Data
(60) Provisional application No. 63/397,833, filed on Aug. 13, 2022.

(51) Int. Cl. A61L 2/10; A61L 2/24

(52) U.S. Cl. CPC A61L 2/10; A61L 2/24; A61L 2202/16; A61L 2202/11; A61L 2202/14; A61L 2202/25

(57) ABSTRACT

A movable disinfectant system used to safely disinfect enclosed areas and/or environments in occupied spaces, such as, e.g., hospitals, medical clinics, administrative or industrial buildings, schools, subways, restaurants, and etc. via filtered far UV-C light irradiation. The disinfectant system comprises a wheeled clamshell tower housing, a plurality of filtered far UV-C lamps with an optical filter housed within a bezel, motion sensors, a 3D or 2D LiDAR scanner, an input interface, a control unit, and a power supply. Far UV-C is a short-wavelength in the ultraviolet C spectrum at 222 nanometers (nm) that is proven safe with human and animal exposure; furthermore, it is more effective for pathogen inactivation than conventional UV-C light. The device is configured to deliver accurate UV-C dosage to effectively and safely inactivate dangerous pathogens in occupied spaces, such as, e.g., on surfaces and in the air.

### CLAIMS OF PRIORITY

This patent application claims priority from:

(1) U.S. provisional patent application No. 63/397,833, entitled ‘Disinfecting system’, filed on Aug. 13, 2022.

The application is incorporated by reference herein in its entirety.

### FIELD OF TECHNOLOGY

This disclosure relates generally to robotic ultraviolet systems and methods for disinfecting an enclosed environment.

### BACKGROUND

Highly infectious pathogens, such as, e.g., bacteria, viruses, spores, and fungi can be found in various indoor environments, such as, e.g., hospitals, schools, offices, and many other public indoor settings and can pose a threat to human health. These dangerous pathogens can cause various diseases and infections such as SARS-CoV-2, influenza, tuberculosis, etc., and can easily spread from person-to-person. Different forms of transmission generally results from direct contact with an infected person within close proximity, physical contact with a contaminated surface and then touching the eyes, nose or mouth, or breathing in infectious droplets in the air into the respiratory tract resulting from the infected person speaking, coughing, or sneezing.

Nosocomial infections, or Hospital-Acquired Infections (HAIs), are infections that a patient contracts in a healthcare setting that were neither present nor developing at the time the patient was admitted. HAIs occur in hospitals, long-term care facilities, medical wards, emergency departments, outpatient clinics, physicians' offices, or community health centers. HAIs can be transmitted from one patient to another through direct or indirect contact. While person-to-person touch is an important mode of transmission, contaminated surfaces in a health care setting can contribute to the transmission of microorganisms causing deadly HAIs. Pathogens can survive on surfaces for long periods and thrive on many objects, such as bed rails, call buttons, telephones, door handles, mattresses, bathroom fixtures, and chairs. For example, C. difficile spores, can survive in the healthcare environment for up to 5 months, and Methicillin-resistant Staphylococcus aureus (MRSA) and Vancomycin Resistant Enterococci (VRE) can survive on dry surfaces for several weeks to months. According to the Centers for Disease Control (CDC), HAIs account for an estimated 1.7 million infections and 99,000 associated deaths each year in American hospitals alone. Other potential risk of exposure includes public areas, such as, e.g., restaurant tables and seating, checkout counters, public transportation seats and rails, card readers, and door handles.

Ultraviolet C (UV-C) disinfection is achieved by utilizing germicidal UV-C irradiation to effectively destroy the pathogens microbial nucleic acids and disrupt their DNA (deoxyribonucleic acid) and RNA (ribonucleic acid), leaving them unable to perform vital cellular functions. This prevents normal proliferation and metabolism of the cells. which causes the microorganisms to die; therefore, is no longer contagious.

UV-C technology has been used in hospitals for decades, but unfortunately, there are major health risks associated with this conventional method of disinfection. Utilizing a short wavelength in the UV-C spectrum at 254 nm (nanometers), it is well known that the irradiation causes erythema to the skin and temporary vision loss with short-term exposure and with long-term exposure, it may lead to skin cancer and cataracts. Due to the dangerous and hazardous effects of conventional UV-C light on humans and animals, disinfection must always be performed in unoccupied spaces. Should there be an operator requiring to operate the UV-C device, the operator must wear personal protective equipment (PPE) which includes goggles, gloves, and a gown to avoid any skin exposure. Additionally, preparing the area or room prior to performing disinfection is necessary, which requires additional manual labor. Glass windows must be covered and gaps between doors or windows must be plugged with cloth or other materials prior to disinfection as UV-C 254 nm wavelength penetrates through glass and irradiates through gaps that can affect people or animals on the opposite side of the room or area. With a breakthrough in UV-C technology recently discovered, we can now mitigate the transmission of dangerous pathogens and promote a healthier and safer environment in occupied spaces.

### SUMMARY

A movable disinfectant system used to safely disinfect enclosed areas and/or environments in occupied spaces, such as, e.g., hospitals, medical clinics, administrative or industrial buildings, schools, subways, restaurants, and etc. via filtered far UV-C light irradiation. The disinfectant system comprises a wheeled clamshell tower housing, a plurality of filtered far UV-C lamps with an optical filter housed within a bezel, motion sensors, a 3D or 2D LiDAR scanner, an input interface, a control unit, and a power supply. Far UV-C is a short-wavelength in the ultraviolet C spectrum at 222 nanometers (nm) that is proven safe with human and animal exposure; furthermore, it is more effective for pathogen inactivation than conventional UV-C light. The device is configured to deliver accurate UV-C dosage to effectively and safely inactivate dangerous pathogens in occupied spaces, such as, e.g., on surfaces and in the air.

The plurality of lamps may radiate light having a wavelength within the range of approximately 190 to 230 nm, such as, e.g., 222 nm. An optical filter may be used to cut off harmful wavelengths outside the range of 190 to 230 nm. With scientific evidence, UV-C irradiation at 222 nm does not penetrate through the stratum corneum, which is the outer most layer of the skin comprising dead cells, nor does it penetrate through the cornea, which is the outer most tear layer of the eye; therefore, it would not cause erythema nor temporary vision loss as conventional UV-C light does. For example, a movable disinfection system comprising filtered far UV-C light, such as, e.g., a KrCl excimer lamp that emits UV-C light having a center wavelength of 222 nm and an optical filter to block the transmission of ultraviolet light having a wavelength lower than 190 nm and higher than 230 nm light may be applied to target areas to safely provide air and surface disinfection of the environment while humans and animals are present. The optical interference filter may comprise a dielectric multilayer film made of SiO₂/Al₂O₃ or SiO₂/MgF₂. The disclosed system attempts to ensure a safer and cleaner indoor environment where people are occupied, reduce the number of HAI infections, mitigate the transmission of dangerous diseases in public indoor settings, and significantly reduce the risk of an outbreak or pandemic.

### BRIEF DESCRIPTION OF THE DRAWINGS

Figures are illustrated by way of example and are not limited to the accompanying drawings, in which, like references indicate similar elements.

FIGS. 1A-E are various views of a disclosed disinfectant system.

FIG. 2 is a block diagram of the disinfectant system.

FIG. 3 is a schematic diagram of a UV-C lamp of the disinfectant system.

FIG. 4A-C are various views of a user interface of the disinfectant system.

FIG. 5 illustrates a spectral distribution curve of an excimer lamp of the disinfectant system.

FIGS. 6A-B are flowcharts of a method for disinfecting a new location.

FIG. 7 is a flowchart for disinfecting an existing location that has already been set up by the system.

FIG. 8 is a table illustrating the amount of time required to disinfect an enclosed area based on the distance of the system to a target area and pathogen type to eradicate.

FIG. 9 is a table illustrating the allowable exposure for human eyes and skin cells.

### DETAILED DESCRIPTION

Although the present has been described with reference to specific examples, it will be evident that various modifications and changes may be made without departing from their spirit and scope. The modifications and variations include any relevant combination of the disclosed features. Equivalent elements, materials, processes or steps may be substituted for those representatively illustrated and described herein. Certain structures and features may be utilized independently of the use of other structures and features. In addition, the components shown in the figures, their connections, couplings, relationships, and their functions, are meant to be exemplary only, and are not meant to limit the examples described herein.

A movable disinfectant system used to safely disinfect enclosed areas and/or environments in occupied spaces, such as, e.g., hospitals, medical clinics, administrative or industrial buildings, schools, subways, restaurants, and etc. via filtered far UV-C light irradiation. The disinfectant system comprises a wheeled clamshell tower housing, a plurality of filtered far UV-C lamps with an optical filter housed within a bezel, motion sensors, a 3D or 2D LiDAR scanner, an input interface, a control unit, and a power supply. Far UV-C is a short-wavelength in the ultraviolet C spectrum at 222 nanometers (nm) that is proven safe with human and animal exposure; furthermore, it is more effective for pathogen inactivation than conventional UV-C light. The device is configured to deliver accurate UV-C dosage to effectively and safely inactivate dangerous pathogens in occupied spaces, such as, e.g., on surfaces and in the air.

The plurality of lamps may radiate light having a wavelength within the range of approximately 190 to 230 nm, such as, e.g., 222 nm. An optical filter may be used to cut off harmful wavelengths outside the range of 190 to 230 nm. With scientific evidence, UV-C irradiation at 222 nm does not penetrate through the stratum corneum, which is the outer most layer of the skin comprising dead cells, nor does it penetrate through the cornea, which is the outer most tear layer of the eye; therefore, it would not cause erythema nor temporary vision loss as conventional UV-C light does. For example, a movable disinfection system comprising filtered far UV-C light, such as, e.g., a KrCl excimer lamp that emits UV-C light having a center wavelength of 222 nm and an optical filter to block the transmission of ultraviolet light having a wavelength lower than 190 nm and higher than 230 nm light may be applied to target areas to safely provide air and surface disinfection of the environment while humans and animals are present. The optical interference filter may comprise a dielectric multilayer film made of SiO₂/Al₂O₃ or SiO₂/MgF₂. The disclosed system attempts to ensure a safer and cleaner indoor environment where people are occupied, reduce the number of HAI infections, mitigate the transmission of dangerous diseases in public indoor settings, and significantly reduce the risk of an outbreak or pandemic.

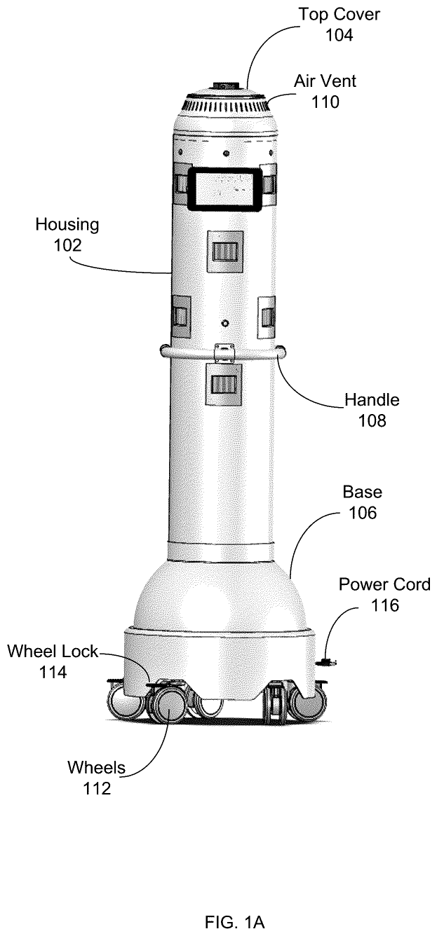

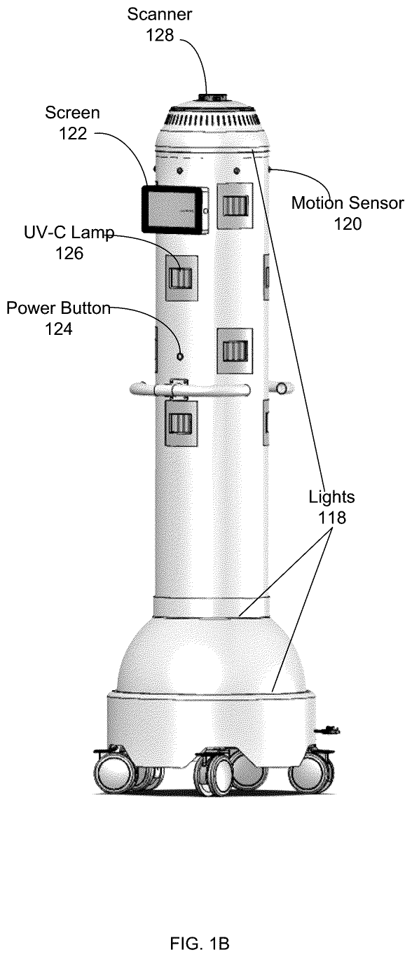

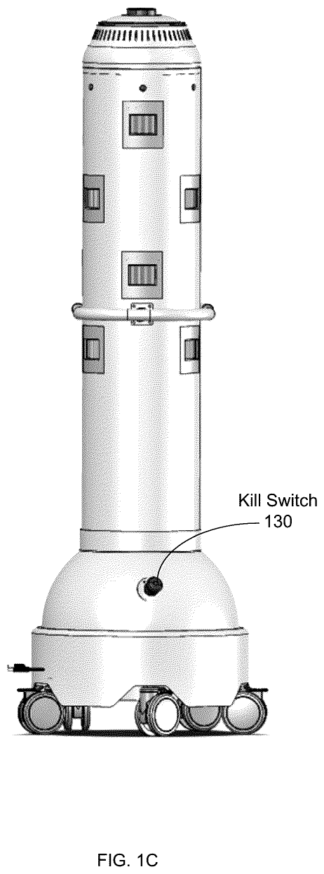

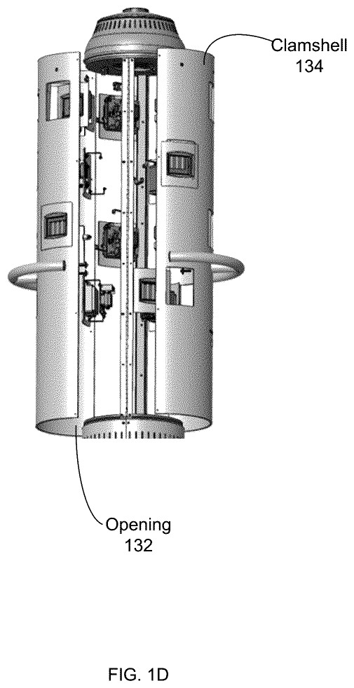

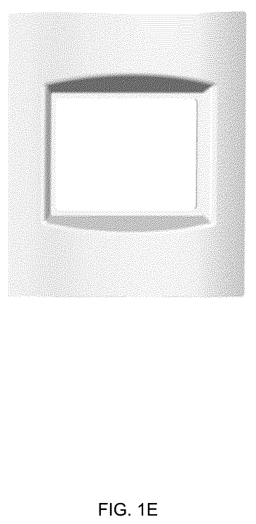

FIGS. 1A-E are various structural views of a disclosed disinfectant system. FIG. 1A is a front view of the apparatus, which includes a freestanding mobile carriage comprising main cylindrical housing 102, top cover 104, and base 106. One or more handle 108 may be attached to a circumference of housing 102 configured for an operator to manually maneuver the apparatus within an enclosed space defined by walls, ceiling and a floor, such as, e.g., a room. Handle 108 may be of circular or semi-circular shape. For example, two semi-circular shape handle 108 may fully encompass housing 102 such that the two ends of a first handle 108 nearly come into physical contact with the other two ends of a second handle 108 that is oriented in the same planar dimension. A spatial gap between handle 108 and housing 102 may permit a human hand to be inserted and to grasp handle 108. There may be one or more sources of UV-C radiation disposed within and around the cylindrical exterior surface of housing 102 to radiate light in a 360-degree manner for maximum coverage. For example, housing 102 may comprise one or more bezels attached to a recess configured to hold the light source, and may be sized and shaped to permit radiation to be projected through an opening of the recess. A plurality of light sources may be positioned at varying heights. The opening may be of any shape, such as, e.g., square, rectangular, circular, or triangular. A reflective material may be located within a portion of the recess such that light that is initially directed away from the target surface emanating from the light source may be reflected and re-directed towards the target surface, such as, e.g., behind the light source. The direct radiation and indirect reflection from the light source may be accomplished at a predetermined intensity and time interval.

Top cover 104 may comprise one or more air vent 110 for dissipating heat from within the apparatus. A blower, such as, e.g., a motorized rotary fan, may be positioned in close proximity to air vent 110 for expelling hot air from within the apparatus and out into the environment through air vent 110. For example, the blower may create negative pressure within housing 102 to draw warm or hot air from around the light source and other electronics out of air vent 110. In some cases, top cover 104 may permit access to internal system components, such as, e.g., a control unit and UV-C lamps.

Base 106 may be disposed at a lower portion of mobile carriage, and may comprise a plurality of omnidirectional wheels 112 positioned in physical contact with a ground for precision moving of the disinfecting apparatus to any desired position in a room, and permitting navigation around furniture and in tight spaces and corners. Wheel lock 114 may be coupled to wheels 112 to prevent movement when it is desired for the apparatus to remain stationary. Power cord 116 may be plugged into a suitable electrical outlet for providing high voltage electricity supply for powering the operations of the disinfectant system. Power cord 116 may be stored on a retractable reel disposed within base 106.

FIG. 1B is an angled view of the disinfectant system. Visible lights 118 may comprise a plurality of indicator and/or work lights disposed at various portions of the apparatus, such as, e.g., top, middle, and bottom portions. Indicator lights 118 may be configured to emit light that conveys information about the status of the system, and can be multi-colored or single-colored. For example, indicator lights 118 may notify a user of the system's on or off status, cycle completion percentage, network connectivity, and other operating parameters. Work lights 118 may be configured to illuminate a target surface to be disinfected, which may be desirable in dark or dimly lit environments. The lights 118 can be LEDs and/or incandescent light sources, with or without lenses.

A plurality of motion sensor 120 may be circumferentially disposed around housing 102 to provide a 360-degree field-of-view around the apparatus. The operator may choose to disable or enable the motion sensor 120. When enabled, the motion sensor 120 may detect movement within a specific distance from the disinfecting system, and to generate an automatic system shut-off signal to a control unit when movement is detected. The control unit may turn off one or more system operations, such as, e.g., UV-C emissions, in response to ensure those who opt out for UV-C exposure does not get exposed within close proximity. In some cases, motion sensor 120 may include laser technology, and in other cases, motion sensor 120 may be an ultrasonic or infrared motion sensor.

A display screen 122, such as, e.g., LCD or LED, fitted with touch screen capability or other input controls may be mounted and slightly protrudes from a front side of housing 102. The display screen 122 may enable a user to interface with the system, such as to manually control various operating parameters, e.g., cycle duration and target dosage. The user graphical interface preferably has capability for being password protected or implements other credential-based login systems that only allow authorized personnel to operate it for programming, repair or diagnostic, or to retrieve data and disinfection history. Power button 124 may be disposed below screen 122, and may be used to turn the system on or off.

One or more filtered far UV-C lamp 126 may be disposed within and around the cylindrical exterior surface of housing 102 to radiate light in a 360-degree manner. For example, housing 102 may comprise one or more bezels attached to a recess configured to hold the light source, and may be sized and shaped to permit radiation to be projected through an opening of the recess without interference with UV lamp 126's field of view. A plurality of filtered far UV-C lamp 126 may be positioned at varying heights. The opening may be of any shape, such as, e.g., square, rectangular, circular, or triangular. A reflective material may be located within a portion of the recess such that light that is initially directed away from the target surface emanating from filtered far UV-C lamp 126 may be reflected and re-directed towards the target surface, such as, e.g., behind filtered far UV-C lamp 126. The direct radiation and indirect reflection from filtered far UV-C lamp 126 may be accomplished at a predetermined intensity and time interval.

In some cases, a plurality of filtered far UV-C lamp 126 may be organized in columns. An operator may manually select a UV-C column to remain off during a disinfection cycle to conserve energy. For example, if the operator chooses to position the device in a corner of a room, the operator may have the option to disable a UV-C column facing a wall to conserve energy and lamp life.

Scanner 128 may include a localization apparatus, such as, e.g., using laser and/or LiDAR technologies, and may be configured to determine the parameters of a room or area and indicate a desired placement of the system within an indoor environment. For example, an ultrasonic wave emitter and return ultrasonic wave sensor can be used to send out ultrasonic waves and then measuring the time it takes for the waves to bounce back to the sensor after hitting an object. By knowing the speed of sound and the time it took for the waves to return, the dimensions of a room and the distances between objects and the device can be calculated. Alternatively, or in addition, LiDAR may be used to measure distances using light, and to generate 3D or 2D models of objects, surfaces, and environments by emitting laser pulses from a sensor or scanner, which bounce off objects and return to the sensor. The time it takes for the light to return to the sensor is used to calculate the distance to the object, while the direction of the laser pulse and the angle at which it was emitted help determine the object's position and orientation in space.

FIG. 1C is a rear view of the disinfectant system. The rear view may comprise a kill switch 130, which may be a safety mechanism used to shut off the apparatus in an emergency when it cannot be shut down in a usual manner, such as, e.g., by depressing power button 124 or via an automatic shut-off mechanism.

FIG. 1D illustrates a clamshell design of the cylindrical housing. Opening 132 may be comprise a bi-valve configured, wherein an external covering that is a two-part hinged shell that opens and closes to permit easy access into the tower for maintenance and services to the components of the device. Dual clamshell 134 may be connected to a plano hinge on one side (hidden from view), which may then be installed onto a fixed aluminum frame disposed inside the housing. The opposite side of the hinge utilizes hardware to secure the clamshell 134 onto the aluminum frame which may be removed to swing one or both clamshells open, exposing the interior of the device.

FIG. 1E shows a bezel of an ultraviolet lamp. The bezel may be configured to house the UV-C lamps onto the clamshell tower, and may be connected to the housing by utilizing a bonding agent to adhere the two surfaces together. The UV-C lamps are then installed onto the bezels with hardware to hold the lamps in place. The bezel may be sized and shaped to permit radiation to be projected through an opening without interference with a UV-C lamp field of view.

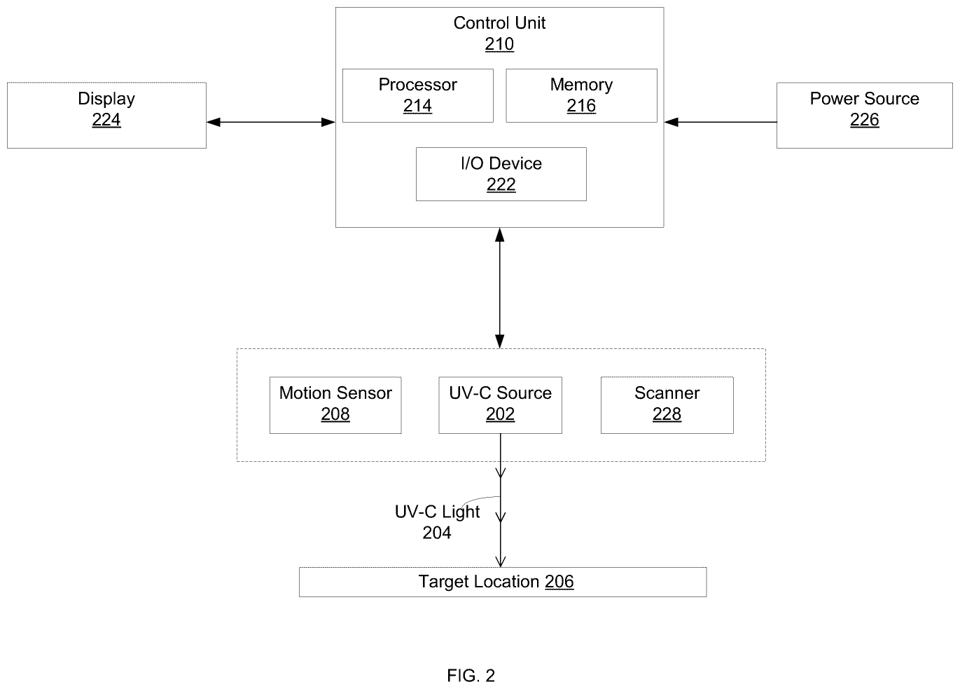

FIG. 2 is a block diagram of the disinfectant system. The apparatus may include a UV-C source 202 configured to emit filtered far UV-C light 204 towards target location 206 to at least partially inactivate airborne and surface-deposited pathogens at target location 206. UV-C source 202 may include, e.g., a UV-C light emitting diode (UV-C LED), a UV-C bulb, or a scanning UV-C laser, and may be a single element or an array of multiple elements. An optical filter may be used to cut off radiation that does not fall within a predetermined wavelength range that is safe to the human skin and eyes. Additionally, UV-C source 202 may include one or more lens and/or mirrors to direct the output energy to target location 206. Target location 206 may be a contact surface such as, e.g., tabletop, chair, mobile device, doorknob, wall, floor, and ceiling, and its surrounding area, such as, e.g., ambient air that is in the path of UV-C light 204. The UV-C source 202 may be configured to operate in the absence of people. For example, a motion detector 208 may be used to shut off the device when movement is registered within the vicinity. The UV-C source 202 may also be configured to operate with people present. For example, the plurality of motion detector 208 may be disabled and disinfection can occur in occupied spaces.

The disinfectant system may include a control unit 210 for controlling system operations, and may comprise configured to monitor the actual dosage, e.g., cumulative dosage, of UV-C light 204 at target location 206 in real-time based on an intensity, e.g., power, of UV-C light 204, cycle temporal duration, and/or a distance between UV-C source 202 and target location 206. Distance between UV-C source 202 and target location 206 may be computed by processor 214 coupled with memory 216 of control unit 210 via an algorithm using data from scanner 228's detection of UV-C light 204 that is reflected from target location 206. Based on the monitored actual dosage of UV-C light 204, control unit 210 may be configured to vary an output power level of UV-C source 202 such that a target dosage matches with the actual dosage. For example, UV-C source 202 may move towards the target location 206, e.g., via an automatic mechanical mechanism such as a piston or manual maneuver by a user, such that the actual dosage at the target location 206 increases. On the other hand, the control unit 210 may reduce the output power of UV-C source 202 or move farther away from target location 206. The system may deactivate UV-C source 202 when the cumulative dosage signal indicates that the target dosage has been achieved. Alternatively, a device configured to detect UV-C light intensity may be positioned on target location 206 for detecting actual dosage of UV-C light 204 exposure at target location 206.

Processor 214 may include one or more processing devices, and memory 216 may include one or more tangible, non-transitory, machine-readable media. By way of example, such machine-readable media can include RAM, ROM, EPROM, EEPROM, or optical disk storage, magnetic disk storage or other magnetic storage devices, or any other medium which can be used to carry or store desired program code in the form of machine-executable instructions or data structures and which can be accessed by processor 214 or by other processor-based devices, such as, e.g., a portable communication device. In some cases, memory 216 is configured to store instructions executable by processor 214 to output various control system signals. For example, processor 214 may execute the control unit 210 instructions to reduce the output power or shut off UV-C source 202, e.g., send a deactivation signal to UV-C source 202 when the cumulative dose signal indicates that the target dosage has been achieved. In some cases, memory 216 may store the history and status of post disinfection cycles, data of whom, when, and where disinfection was performed, and whether the disinfection cycle was successful or unsuccessful.

Control unit 210 may be configured to receive operator input via I/O device 222, such as, e.g., a keyboard, mouse, or touch screen. Display 224, such as, e.g., a computer monitor or screen, configured to display information related to one or more operation of the UV-C disinfectant system. Power source 226 may provide electrical supply for powering the operations of the device, and may include, e.g., capacitors, inductors, resistors, and/or a transformer.

Scanner 228 may include a localization apparatus, such as, e.g., using ultrasonic and/or LiDAR technologies, and may be configured to indicate a desired placement of the system within an indoor environment. For example, an ultrasonic wave emitter and return ultrasonic wave sensor can be used to send out ultrasonic waves and then measuring the time it takes for the waves to bounce back to the sensor after hitting an object. By knowing the speed of sound and the time it took for the waves to return, the dimensions of a room and the distances between objects and the device can be calculated. Alternatively, or in addition, LiDAR may be used to measure distances using light, and to generate 3D or 2D models of objects, surfaces, and environments by emitting laser pulses from a sensor or scanner, which bounce off objects and return to the sensor. The time it takes for the light to return to the sensor is used to calculate the distance to the object, while the direction of the laser pulse and the angle at which it was emitted help determine the object's position and orientation in space.

### --Optimal Position, Inventive--

In some cases, data from scanner 228 is fed into control unit 210 for determining the parameters of the room/area and determine an optimal placement of the system within a site. The optimal placement is calculated to enable the disinfectant system to deliver efficient dosage of UV-C radiation, based on the room size and selected pathogen the user chooses to target. The selection of pathogens, such as, e.g., virus, bacteria, and fungi, may be preset, and may be based on species type. Display 224 may show a user the optimal position and placement within the site. The desired placement may depend on a shape and a size of the enclosed space, e.g., square footage of the enclosed space when targeting surface locations, and cubic dimension of the enclosed space when targeting airborne pathogen. For example, once the parameters, such as, e.g., square footage, of a room or area is identified by a LiDAR scanner, control unit 210 creates a convex hull of the measured site. The information is then transferred to display 224, illustrating the layout of the room or area, and the calculated optimal position to place the disinfection device. Depending on the parameters and/or layout of the room or area, multiple positions or placements may be required and calculated. Square footage is a measurement of the area of the enclosed site, and is represented by the product of its length and width. Cubic dimension refers to the three-dimensional measurement of a space in terms of length and width which is measured by the LiDAR scanner, and height, which may be manually inputted into the system by the user, and is represented as the product of these three dimensions. The device may then be manually maneuvered to the location once identified by the system.

### --Disinfection Cycles, Inventive--

The disinfectant system may comprise a plurality of disinfection cycles stored within control unit 210 from initial setup of each room or area. Interface 220 may be configured to receive a selection of any disinfection cycle from the stored room or area in the database and may relay the input to control unit 210. Control unit 210 may power the UV-C source 202 corresponding with the chosen room or area in the database and selected pathogen to target, which will determine the disinfection cycle. A disinfection cycle may be defined by localization data obtained from scanner 228, such as, e.g., an area's square footage, and the selected pathogen, to determine the required temporal duration for the cycle.

Control unit 210 may be configured to automatically shut off the disinfecting system once a cleaning cycle is complete, or when there is an automatic shut-off signal. For example, motion sensor 208 may be configured to monitor motion proximate target location 206 and output motion data to control unit 210. Based at least in part on the motion data, controller 210 may determine whether a person is disposed in a path of motion sensor 208. In response to detecting a person in the path of motion sensor 208, control unit 210 may output a deactivation signal to deactivate UV-C source 202. In some cases, UV-C source 202 may automatically re-activate in response to control unit 210 determining that the person is no longer in the path of motion sensor 208, such as, e.g., due to not receiving motion data from motion sensor 208 for a predetermined amount of time. Resuming the disinfection cycle avoids restarting the interrupted disinfection cycle from the beginning and saves a significant amount of time. Alternatively, the device may require manual re-activation of UV source 202. Further, in response to detecting a person in the path of motion sensor 208, control unit 210 may also output a warning sound signal configured to provide a visual cue that UV-C source 202 has been deactivated in response to detecting a person in the path of UV-C source 202.

### --Inventive--Continuous Disinfection Mode--

In some cases, a continuous disinfection cycle may be used to intermittently and automatically turn the filtered UV-C source 202 on and off throughout an 8-hour period. For example, after 8 hours have been reached, the device may turn off automatically and display 224 may show that the continuous cycle has been completed. The continuous disinfection cycle may start a new cycle immediately after one has been completed, or the system may run the continuous disinfection cycle for a predetermined duration manually set by the user. If the device is being used in the same room or area, the user may restart another continuous disinfection cycle in increments of 8 hours. The duration of how long UV-C source 202 stays on and off may be based on the room size in order to ensure proper UV-C dosage for effective disinfection. This duration may be calculated using localization data from scanner 228, or may be set manually by an operator.

As set forth above, UV-C source 202 may be configured to automatically provide dosage of UV-C irradiation 204 to target location 206 in order to achieve effective inactivation of pathogens on a surface or in the environment of target location 206, in addition to airborne pathogens in the path of UV-C light 204 based on the room or area size determined by the LiDAR scanner and the selected target pathogen by the user. For example, the target dosage may be less than 100 mJ/cm², such as, e.g., 4 or 9 mJ/cm², of UV-C light 204 to target location 206. To achieve the target dosage, the disinfectant device may be positioned at an optimal positon within an enclosed area, such as, e.g., a room, before activating UV-C source 202 for a disinfection cycle lasting a predetermined amount of time, as discussed in detail above. A target dosage of UV-C light 204 may be computed by control unit 210, and may be based on localization data obtained by scanner 228, such as, e.g., cubic dimensions of the enclosed area. Cubic dimension refers to the three-dimensional measurement of a space in terms of length and width which is measured by the LiDAR scanner, and height, which may be manually inputted into the system by the user, and is represented as the product of these three dimensions. Once the duration of the calculated disinfection cycle has been completed to target location 206, control circuit 210 may deactivate UV-C source 202.

### --Inventive, Lamp Monitor--

Further, the disinfectant system may comprise a lamp life monitoring apparatus configured to monitor total number of hours each UV-C source 202 have been operated to indicate end of lamp life. The apparatus may include an electrical wire coupled to an instrument configured for measuring electrical output, such as e.g., a voltmeter, and connected to UV-C source 202 for monitoring electrical current output of UV-C source 202. Each UV-C source 202 of a plurality of UV-C source 202 may include its own electrical wire coupled to a single voltmeter or separate individual voltmeters. The single voltmeter may take separate readings of each UV-C source 202.

For example, the device may be able to monitor the functionality of each lamp as well as the total runtime of each lamp individually. A color coded indicator may display a green circle to show that the lamp is functional. If a lamp is not working, the green circle may change to red which indicates that the specific lamp is non-functional and will prompt the operator to replace the lamp. The system may also monitor the total runtime of each lamp individually. For instance, if a lamp burns out or is malfunctioning, it will have a lower runtime compared to the rest of the lamps. Once it is replaced, the runtime will start at 00:00:00 where the rest will continue to record the total runtime.

In some cases, the monitoring apparatus is configured to generate a signal indicative of a malfunction of any of a plurality of UV-C source 202. Control unit 210 may receive the malfunction signal and send a notification to the user to prompt a replacement of the UV-C lamp and automatically calculate the required UV-C irradiance, or intensity, that is required to achieve the target dosage from the rest of UV-C source 202 based on data from the malfunctioning UV-C source 202 and the chosen duration cycle. For example, control unit 210 may direct a functioning UV-C source 202 of a plurality of UV-C source 202 to increase the duration of the disinfection cycle due to another UV-C source 202's malfunctioned status in order to achieve the target dosage for the room or area. Further, varying durations of each disinfection cycle increases may be based on varying levels of lamp degradation or malfunction, e.g., one or more UV-C source 202 may output a cumulative 20% increased duration of the chosen cycle when a UV-C source 202 of the plurality of UV-C source 202 is degraded by 20%. This is particularly important because once the quality of a lamp has been degraded, it will lose effectiveness to disinfect, unless other lamps within the system makes up for the loss. The data generated by the lamp life monitoring apparatus, such as, e.g., UV-C source 202's functional status, electrical current output, and lamp operational duration, may be communicated to display 224, and may be viewed by an operator.

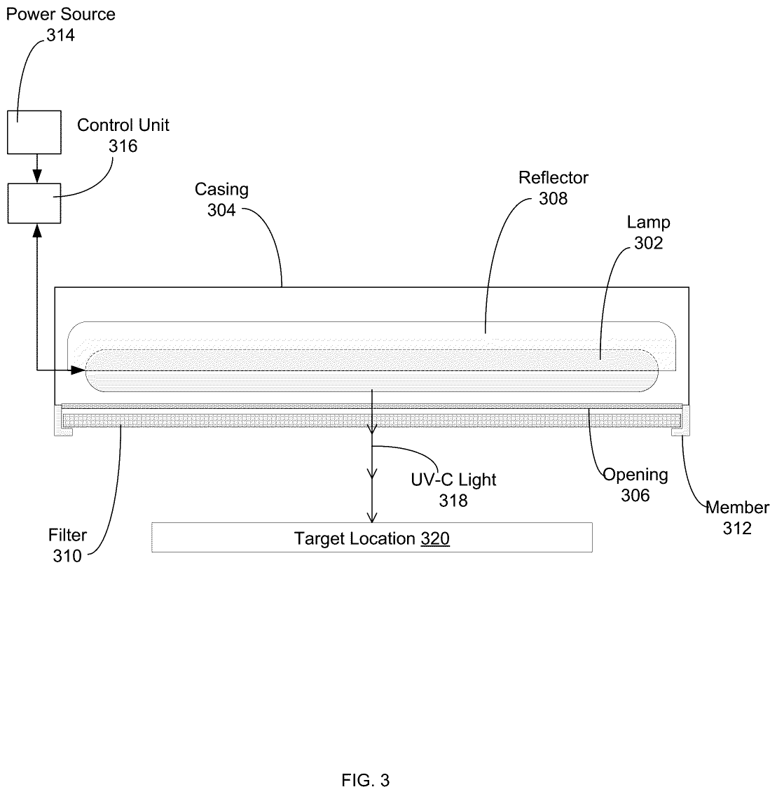

FIG. 3 is a schematic diagram of a UV-C lamp of the disinfectant system. A rod-shaped excimer lamp 302 may be disposed within protective casing 304, which in turn may be disposed within a recess of the disinfectant system's housing. Lamp 302 may, for example, be an LED using a nitride semiconductor. Casing 304 may have a rectangular solid outer shape; however, it may take any other form, such as, e.g., square, circular, or triangular. An ultraviolet transmission opening 306 of rectangular plate shape made from, e.g., synthetic quartz glass, may be provided on a lower side of casing 304. Lamp 302 may oppose opening 306 on an inside portion of casing 304. A parabolic reflector 308 for reflecting light emissions from lamp 302 toward opening 306 is arranged behind lamp 302 in casing 304 such that reflector 308 surround a portion of lamp 302 that is farthest from opening 306. Reflector 308 may be made from a metallic material, such as, e.g., stainless steel for aluminum; however, in some cases, Polytetrafluoroethylene (PTFE) may be used. To prevent intensity attenuation of the light from excimer lamp 302, interior of casing 304 may be purged with inert gas, such as, e.g., nitrogen gas. An optical filter 310 of rectangular plate shape may be arranged outside casing 304 at a side of opening 306 that is farthest from reflector 308. Filter 310 may be fixed to casing 304 by a pair of fixing member 312, and may comprise a dielectric multilayer film including SiO₂ films and MgF₂ films on both sides of a substrate made of synthetic quartz glass, in order to transmit light having a wavelength within a range of 190 to 230 nm, and to cut out radiation below 190 nm and above 230 nm. A power source 314 configured to supply electricity to lamp 302 is coupled to lamp 302 through electronic control unit 316. For example, controller 316 may limit the output of lamp 302 to less than 100 mJ/cm², such as, e.g., 4 or 9 mJ/cm².

In the foregoing disinfection apparatus, UV light 318 from excimer lamp 302 is emitted out of the casing 304 through opening 306 to disinfect target location 320 via optical filter 310. For example, a KrCl excimer lamp that emits light having a center wavelength of 222 nm may be used. The device may kill or inactivate pathogens, such as, e.g., bacteria, virus, and fungi that are present on target location 320 and within a line of travel of UV-C light 318, while suppressing damage or human cells.

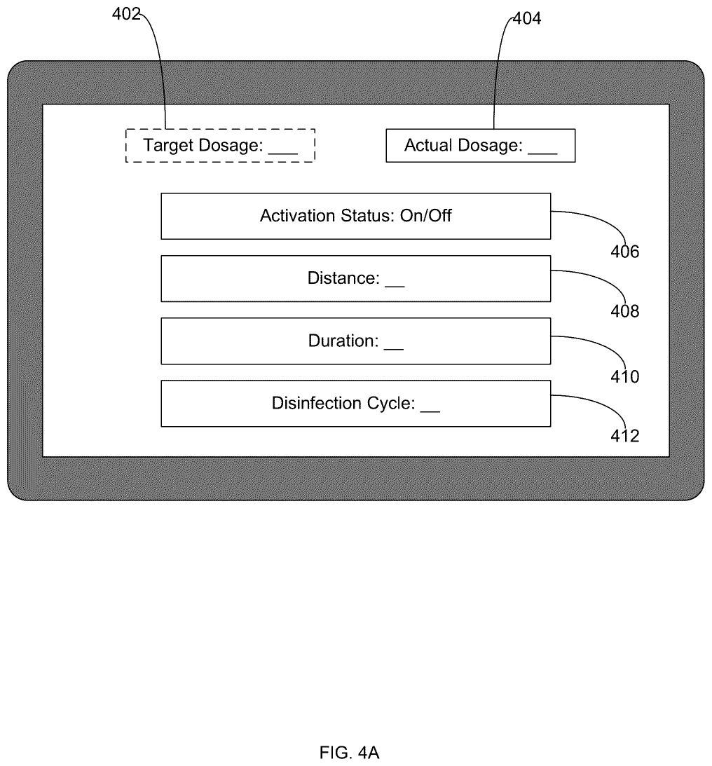

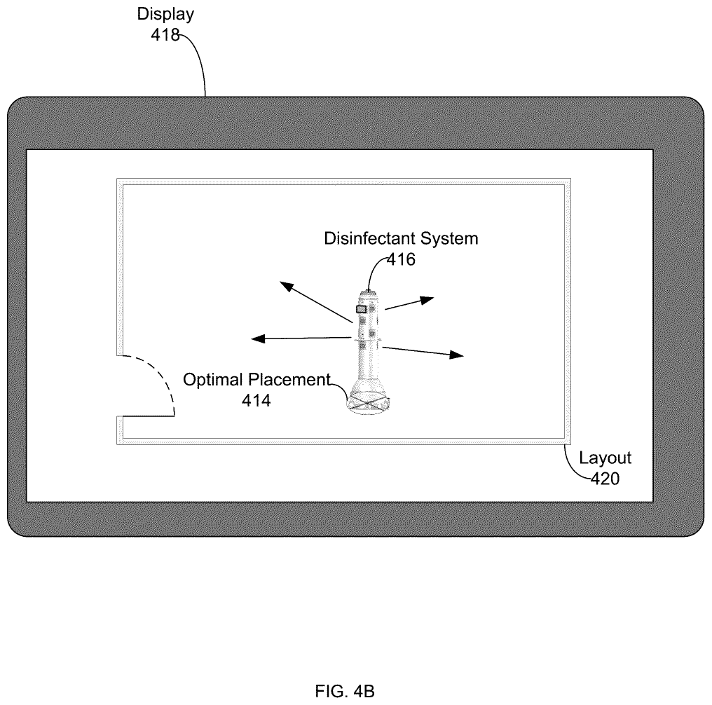

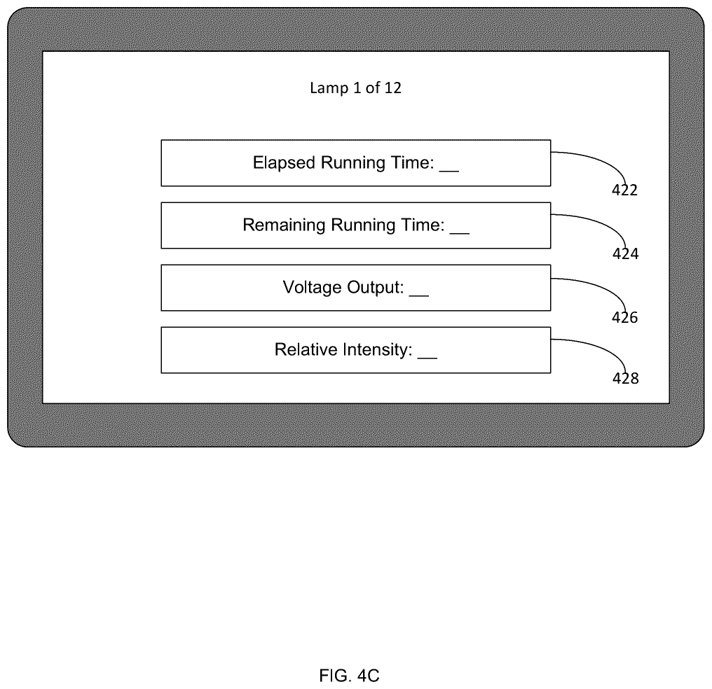

FIG. 4A-C are various views of a user interface of the disinfectant system. In FIG. 4A, the user interface displays operational data of a disinfection cycle of the system. The display may be configured to receive operator input through an input/output device, such as, e.g., a keyboard, mouse, or touch screen, configured to provide user input to a control unit. The user interface may be shown on a display, such as, e.g., a computer monitor or screen. Frame 402 may display a target dosage amount that can be adjusted by the operator, or may be a preset value based on a disinfection cycle, which in turn may be based on the room or area. For example, the operator may tap on the icon corresponding to frame 402 to modify the target dosage. Frame 404 is the actual, cumulative, dosage amount that is computed by a dosimetry circuit coupled with the control unit. The system may be configured to match the value of frame 402 with the value of frame 404 in order to successfully complete a disinfection cycle. Frame 406 may display an activation status, which indicates whether one or more UV-C light source is powered on or off. The operator may tap on the icon corresponding to frame 406 to manually power the system on or off. An automatic mechanism may also power the system on or off, such as, e.g., initiation or completion of a disinfection cycle, a motion sensor detects movement within the disinfection site, and when the target dosage matches with the actual dosage. In some cases, processor executable instructions configured to activate various components before, during, or after activation or deactivation of the UV-C light source may be initiated, such as, e.g., activation or deactivation of the visible light. Frame 408 indicates the distance measured between the disinfectant system and a target location to be disinfected, as measured by a distance sensor. The distance may be adjusted based on a manual maneuvering of the device. Frame 410 displays a timer shown as either an elapsed amount of time or a remaining time for the disinfection cycle in process. In some cases, moving the disinfectant apparatus, e.g., changing the distance between the system and the target location, may cause the timer of frame 410 to increase or decrease accordingly. For example, moving the apparatus farther away from the target location may cause the timer to increase, e.g., unless intensity of the UV-C light source is also increased. Frame 412 may indicate a disinfection cycle that was selected by the operator. The operator may press the icon corresponding to frame 412 to bring up a menu with pre-programmed cycles to choose from. In some cases, the preset disinfection cycles are based on a room or area that has been scanned by a localization apparatus.

FIG. 4B shows the disinfectant system positioned in an optimal position within an enclosed space. The enclosed space may be defined by walls, ceiling, and floor, such as, e.g., a room within a building. Data from a localization apparatus, such as, e.g., a LiDAR or ultrasonic scanner, is fed into a control unit for determining a desired placement 414 of the system within the site. The desired placement 414 is calculated to enable disinfectant system 416 to deliver accurate efficient dosage of UV-C irradiation, based on the room size and selected pathogen the user chooses to target. The selection of pathogens, such as, e.g., virus, bacteria, and fungi, may be preset, and may be based on species or variant type. Display 418, such as, e.g., a monitor or screen, may show the optimal position and placement 414 within the site. The desired placement may depend on square footage of the enclosed space when targeting surface locations, and may depend on cubic dimension of the enclosed space when targeting airborne pathogen. For example, once the parameters, such as, e.g., square footage, of a room or area is identified by a LiDAR scanner, the control unit creates a convex hull of the measured site. The information is then transferred to display 418, illustrating layout 420 of the room or area, and the calculated optimal placement 414 to place the disinfection device. Depending on the parameters, and/or layout of the room or area, multiple positions or placement 414 may be calculated. Square footage is a measurement of the area of the enclosed site, and is represented by the product of its length and width. Cubic dimension refers to the three-dimensional measurement of a space in terms of length and width which is measured by the LiDAR scanner, and height, which may be manually inputted into the system by the user, and is represented as the product of these three dimensions. The device may then be manually maneuvered to the location once identified.

In some cases, a plurality of filtered far UV-C lamps may be organized in columns. An operator may manually select a UV-C column to remain off during a disinfection cycle to conserve energy. For example, if the operator chooses to position the device in a corner of a room, the operator may have the option to disable a UV-C column facing a wall to conserve energy and lamp life.

FIG. 4C is a user interface of a lamp monitoring mechanism of the disinfectant system. Each of a plurality of UV-C lamp may be coupled to an electrical output measuring device, such as, e.g., a voltmeter, via an electrical wire. The voltmeter may be coupled to a control unit. Frame 422 may display an elapsed running time of a lamp, indicating the total cumulative amount of time that the lamp has been in operation. Frame 424 may indicate a remaining running time left on the lamp. When the remaining running time approaches zero, the signal may alert an operator to change the lamp for another lamp with a non-zero remaining running time. The total amount of running time that a lamp is set to may be based on, e.g., the disinfectant system's manufacturer recommendation, the lamp manufacturer's recommendation, or an operator's preference. In addition, the monitoring mechanism may alert the operator if the lamp is malfunctioning, e.g., if it is not working properly not due to the remaining running time approaching zero. Frame 426 tracks the lamp's voltage output, such that if the electric current decreases to a predetermined threshold, a malfunctioning signal may be generated. A control unit may receive the malfunction signal and automatically calculate the required UV-C irradiance, or intensity, that is required to achieve a target dosage from the rest of the lamps based on data from the malfunctioning lamp and the chosen duration cycle. For example, the control unit may direct a functioning lamp of a plurality of UV-C lamps to output a higher intensity value due to another UV-C source 202's malfunctioned status in order to achieve the target dosage within the duration cycle computed by control unit 210. Further, varying levels of intensity increases may be used based on varying levels of lamp degradation, e.g., one or more lamps may output a cumulative 20% increased intensity value over the duration of the chosen cycle when a lamp of the plurality of lamps is degraded by 20%. This is particularly important because once the quality of a lamp has been degraded, it will lose effectiveness to disinfect, unless other lamps within the system makes up for the loss. Frame 428 may display a relative intensity of the lamp such that a 100% relative intensity signifies that it is a brand new lamp with 100% operational functionality. A decrease in the displayed percentage shows the gradual degradation in the UV-C lamp's output compared to its output when in a new condition. The user can set a low-level threshold of UV-C being emitted to ensure the proper dosage for an effective disinfection. In order to operate the disinfectant system efficiently and to obtain a consistently good curing result, careful monitoring of the UV-C output is performed. If the lamps are only replaced at set time intervals, they may be replaced too soon or too late, creating needless waste of resources.

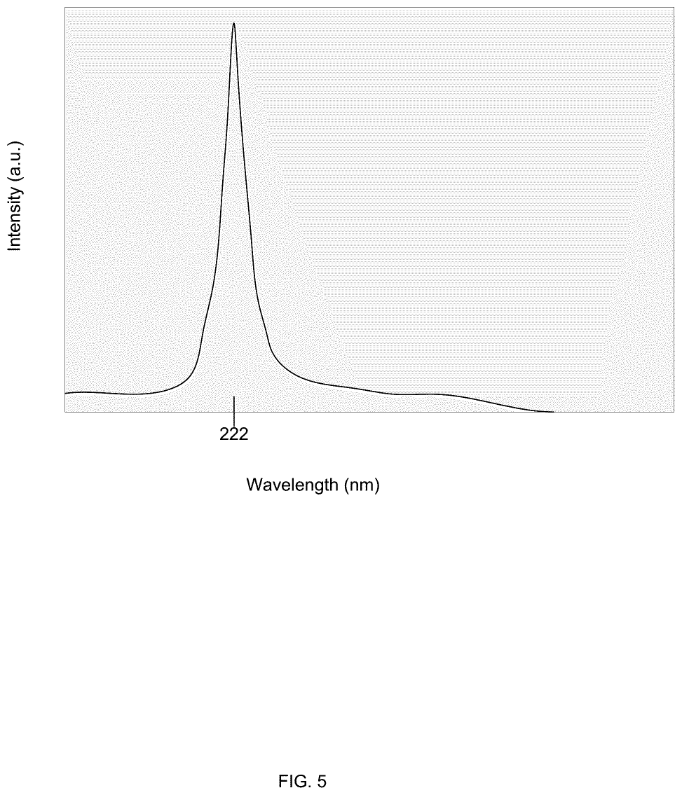

FIG. 5 illustrates a spectral distribution curve of an excimer lamp of the disinfectant system. The plurality of lamps may radiate light having a wavelength within the range of approximately 190 to 230 nm. An optical filter may be used to cut off light having a wavelength outside the range. For example, UV-C light having a wavelength of 190 to 230 nm is applied to a disinfection target surface with the use of a light source, such as, e.g., a KrCl excimer lamp that emits light having a center wavelength of 222 nm, and an optical filter for blocking transmission of ultraviolet light having a wavelength of lower than 190 nm and more than 230 nm. A power supply unit may be controlled by a control unit such that the irradiation amount of the light having a wavelength within the range of 190 to 230 nm is not more than 100 mJ/cm², such as, e.g., 4 or 9 mJ/cm².

Skin cancer occurring when human skin is irradiated with ultraviolet rays is caused as a result of damage to DNA of the skin cells from the ultraviolet rays. For example, if the skin is irradiated with ultraviolet rays in a wavelength range including 260 nm, bases constituting DNA in the skin cells are excited. In the process of returning to the ground state, the bases react with each other to generate dimer molecules such as cyclobutane pyrimidine dimer (CPD) and 6-4PP. Such damage to DNA causes a change in the DNA structure, whereby DNA replication and RNA transcription are hindered.

In such ultraviolet-based generation of skin cancer, the irradiation amount, or dosage, of ultraviolet rays depends on a threshold amount for a specific wavelength or range. Continuous exposure to ultraviolet rays included in sunlight causes a change in skin, such as erythema. More specifically, if skin is irradiated with the ultraviolet rays, erythema occurs according to the irradiation amount. In order to avoid the risk of developing a skin disease by the ultraviolet irradiation, the irradiation amount of the ultraviolet rays is usually set below the value of minimal erythema dose (MED).

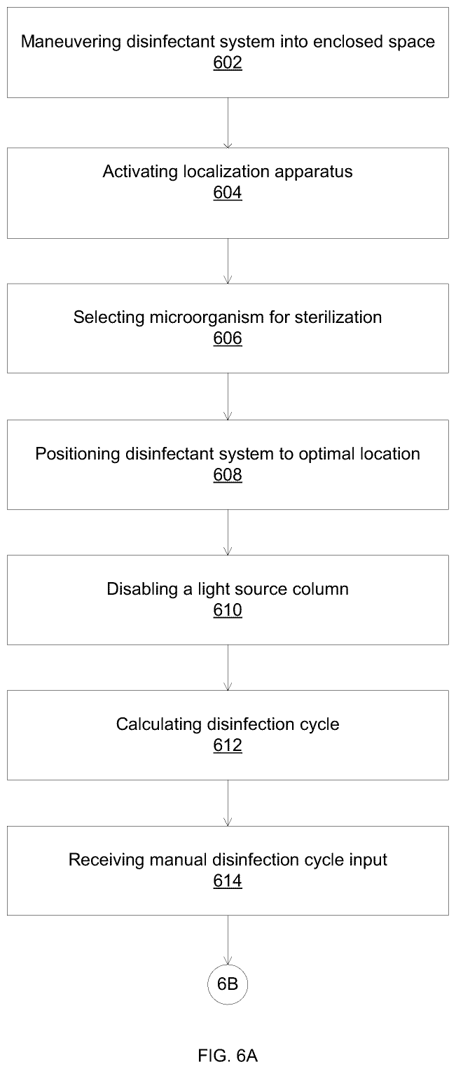

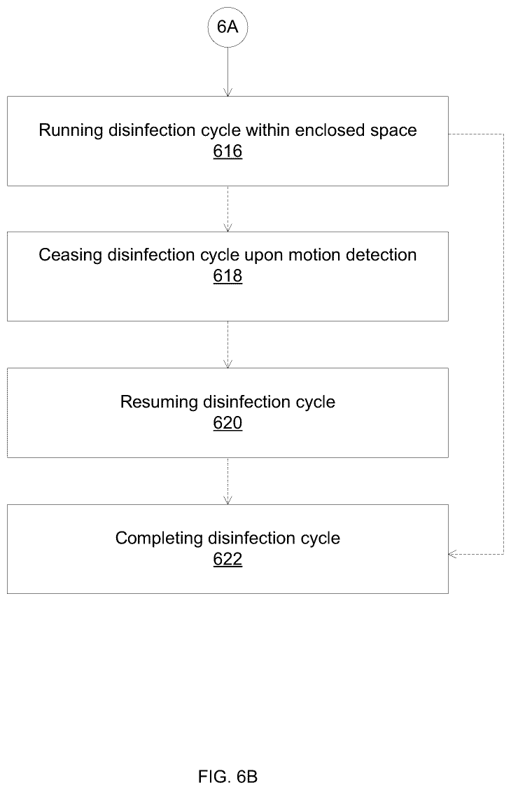

FIGS. 6A-B are flowcharts of a method for disinfecting a new location. The new location may require an initial set up phase, as will be described hereafter. Operation 602 manually maneuvers the disinfectant system into an enclosed space with one or more target locations to be disinfected, such as, e.g., a room of a building. For example, an operator may grasp handles attached to the apparatus and wheel it to a destination. Operation 604 activates a localization apparatus for an initial set up of an enclosed space, such as, e.g., LiDAR or ultrasonic scanner for determining an optimal placement of the disinfectant system. The placement is calculated based on room size, such as, e.g., square footage or cubic dimension, to enable the disinfectant system to deliver efficient dosage of UV-C radiation, so as to kill any undesirable pathogen. The optimal placement may depend on shape and size of the enclosed space, such as, e.g., square footage of the enclosed space when targeting surface locations, and may depend on cubic dimension of the enclosed space when targeting airborne pathogen. Operation 606 selects the microorganism for disinfection. The pathogen type may be indicated by species or variant type, such as, e.g., SARS-CoV-2 (B1), SARS-CoV-2 (Delta), Influenza (H1N1), MRSA, etc. Operation 608 positions the disinfectant system to the one or more optimal placement location within the enclosed space, shown on a display of the apparatus. Operation 610 optionally disables a UV-C light source column. For example, if the optimal placement of the device is in a corner of a room, the operator may have the option to disable a UV-C column facing a wall to conserve energy and lamp life. Operation 612 automatically calculates a disinfection cycle calculated by a control unit, which may be based on the optimal position, room size, and selected pathogen type. Operation 614 optionally receives a disinfection cycle input for the room or area by the operator. The operator input may be accomplished through an input interface, such as, e.g., a touchscreen, keyboard, or mouse. The automatically generated disinfection cycle and the manually inputted disinfection cycle may be saved in the control unit.

Operation 616 runs a disinfection cycle within the enclosed space. The chosen disinfection cycle may be through an automatic or manual mechanism, as discussed. In some cases, the disinfection cycle may be a continuous cycle that operates continuously for a predetermined amount of time, such as, e.g., 8 hours. Optionally, a target dosage may be manually adjusted by the operator. Operation 618 ceases the disinfection cycle upon sensing motion within the enclosed space. Operation 620 resumes the disinfection cycle at the point of cessation when motion is no longer detected for a predetermined amount of time. Operation 622 shuts down the disinfectant system upon completion of the disinfection cycle, e.g., actual dosage matches with target dosage or after completing the duration of the disinfection cycle. In some cases, if no motion is detected by the motion sensor, the system may proceed from running the disinfection cycle in the enclosed space of operation 616 directly to completion of operation 622, effectively bypassing operations 618 and 620.

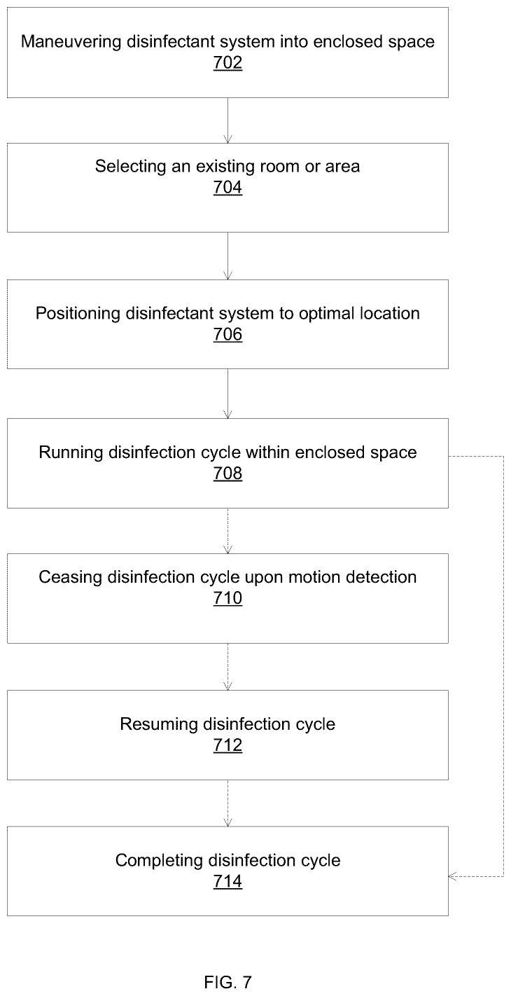

FIG. 7 is a flowchart for disinfecting an existing location that has already been set up by the system. Operation 702 manually maneuvers the disinfectant system into an enclosed space with one or more target locations to be disinfected, such as, e.g., a room of a building. For example, an operator may grasp handles attached to the apparatus and wheel it to a destination. Operation 704 selects an existing room or area that is stored in a control unit, and to be disinfected. The existing room or area may comprise preset disinfection cycles. Operation 706 positions the disinfectant system to the one or more optimal placement location within the enclosed space, shown on a display of the apparatus. Operation 708 runs a disinfection cycle within the enclosed space. The chosen disinfection cycle may be through an automatic or manual mechanism. In some cases, the disinfection cycle may be a continuous cycle that operates continuously for a predetermined amount of time, such as, e.g., 8 hours. Optionally, a target dosage may be manually adjusted by the operator. Operation 710 ceases the disinfection cycle upon sensing motion within the enclosed space. Operation 712 resumes the disinfection cycle at the point of cessation when motion is no longer detected for a predetermined amount of time. Operation 714 shuts down the disinfectant system upon completion of the disinfection cycle, e.g., actual dosage matches with target dosage or after completing the duration of the disinfection cycle. In some cases, if no motion is detected by the motion sensor, the system may proceed from running the disinfection cycle in the enclosed space of operation 708 directly to completion of operation 714, effectively bypassing operations 710 and 712.

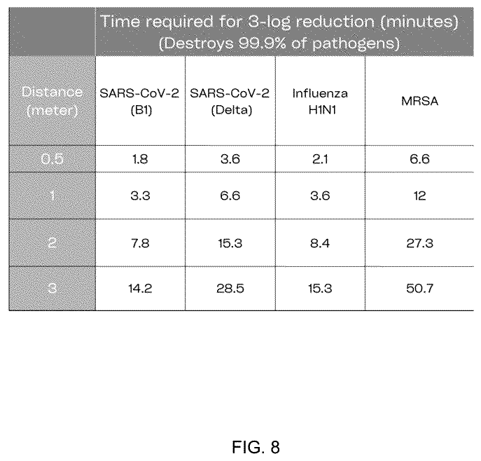

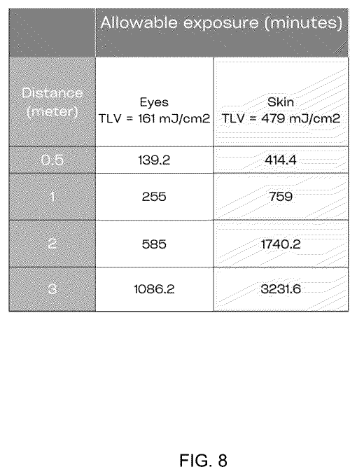

FIG. 8 is a table illustrating the amount of time required to disinfect an enclosed area based on the distance of the system to a target area and pathogen type to eradicate. The pathogen may be specified by species or variant, such as, e.g., SARS-CoV-2 (B1), SARS-CoV-2 (Delta), Influenza (H1N1), and MRSA. Optimal placement of the disinfection device may depend on room size and shape. For example, at a distance of 1 m, the device may run a disinfection cycle that lasts at least 3.3 minutes in order to destroy 99.9% of the SARS-CoV-2 microorganisms that are present in a target location.

FIG. 9 is a table illustrating the allowable exposure for human eyes and skin cells. For example, the human eyes may have a threshold limit value (TLV) of 161 mJ/cm 2. As such, at a distance of 1 m, the allowable duration of UV-C exposure to the human eyes is 255 minutes.

A number of examples have been described. Nevertheless, it will be understood that various modifications may be made without departing from the spirit and scope of the claimed invention. In addition, the logic flows depicted in the figures do not require the particular order shown, or sequential order, to achieve desirable results. Other steps may be provided, or steps may be eliminated, from the described flows, and other components may be added or removed. Accordingly, other examples are within the scope of the following claims.

What is claimed is:

1. A disinfectant method, comprising:
maneuvering an ultraviolet irradiation disinfectant system into an enclosed space,
wherein the disinfectant system comprises a cylindrical housing and a plurality of omnidirectional wheels,
wherein the cylindrical housing comprises a bivalve opening,
wherein the bivalve opening permits access to the disinfectant system's internal components,
wherein the internal components comprise a UV-C light source and an optical filter,
wherein the UV-C light source emits radiation at 222 nm wavelength,
wherein the enclosed space comprises at least one target location to be disinfected;
activating a localization apparatus for determining an optimal placement of the disinfection system within the enclosed space;
selecting a target microorganism for disinfection; and
positioning the disinfectant system to the optimal placement within the enclosed space.

2. The disinfectant method of claim 1, further comprising:
calculating one or more disinfection cycle based on the selected microorganism and a size of the enclosed space.

3. The disinfectant method of claim 1:
wherein the optical filter comprises a dielectric multilayer film made of SiO₂/Al₂O₃ or SiO₂/MgF₂, and
wherein the optical filter blocks radiation below 190 nm and above 230 nm.

4. The disinfectant method of claim 1:
wherein the UV-C light source and optical filter are disposed within a bezel of the cylindrical housing.

5. The disinfectant method of claim 1:
wherein the UV-C light source is a KrCl excimer lamp.

6. The disinfectant method of claim 1:
wherein the at least one target location to be disinfected is at least one of a surface location and an airborne location.

7. The disinfectant method of claim 1:
wherein the optimal placement is based on at least one of room size and room shape and a selected target microorganism,
wherein room size equates to square footage when the target location is a surface location, and
wherein room size equates to cubic dimension when the target location is an airborne location.

8. The disinfectant method of claim 1, further comprising:
adjusting a target dosage of the disinfection cycle, and
wherein the target dosage is based on a cubic dimension of the enclosed space.

9. The disinfectant method of claim 1, further comprising:
receiving a manual disinfection cycle input by an operator of the disinfection system.

10. The disinfectant method of claim 9:
wherein the disinfection cycle is a continuous cycle with a customized duration interval set by the operator, and
wherein a continuous cycle is in increments of 8 hours.

11. The disinfectant method of claim 2, further comprising:
running a disinfection cycle within enclosed space.

12. The disinfectant method of claim 11, further comprising:
ceasing the disinfection cycle upon motion detection within the enclosed space.

13. The disinfectant method of claim 12, further comprising:
resuming the disinfection cycle at the point of cessation when motion is no longer detected for a predetermined amount of time.

14. The disinfectant method of claim 13, further comprising:
shutting down the disinfection cycle upon completion a duration of the disinfection cycle or a match of an actual dosage with the target dosage.

15. The disinfectant method of claim 11, further comprising:
increasing a disinfection cycle duration based on a malfunctioning of one or more UV-C light source of a plurality of UV-C light sources.

16. The disinfectant method of claim 1:
wherein a plurality of UV-C light sources is arranged in a columnar configuration.

17. The disinfectant method of claim 1:
wherein the system is next to a wall or object of the enclosed space, and
disabling a UV-C light source column juxtapose the wall or object of the enclosed space.

18. The disinfectant method of claim 17, further comprising:
monitoring a functionality and a total runtime of the UV-C light source; and
broadcasting the functionality based on a color coding of an indicator lamp.

19. A disinfectant method, comprising:
maneuvering an ultraviolet irradiation disinfectant system into an enclosed space,
wherein a UV-C light source emits radiation at 222 nm wavelength,
wherein the enclosed space comprises at least one target location to be disinfected;
selecting a target microorganism for disinfection;
positioning the disinfectant system to the optimal placement within the enclosed space;
running a disinfection cycle within enclosed space; and
shutting down the disinfection cycle upon completion a duration of the disinfection cycle or a match of an actual dosage with the target dosages.

20. A disinfectant method, comprising:
maneuvering an ultraviolet irradiation disinfectant system into an enclosed space,
wherein the disinfectant system comprises a cylindrical housing and a plurality of omnidirectional wheels,
wherein the cylindrical housing comprises a bivalve opening,
wherein the bivalve opening permits access to the disinfectant system's internal components,
wherein the internal components comprise a UV-C light source and an optical filter,
wherein the UV-C light source emits radiation at 222 nm wavelength,
wherein a plurality of UV-C light sources is arranged in a columnar configuration.
wherein the enclosed space comprises at least one target location to be disinfected;
selecting a target microorganism for disinfection;
activating a localization apparatus for determining an optimal placement of the disinfection system within the enclosed space;
positioning the disinfectant system to the optimal placement within the enclosed space,
wherein the system is next to a wall or object of the enclosed space;
disabling a UV-C light source column juxtapose the wall or object of the enclosed space;
calculating one or more disinfection cycle based on the selected microorganism and a size of the enclosed space,
wherein the optical filter comprises a dielectric multilayer film made of SiO₂/Al₂O₃ or SiO₂/MgF₂,
wherein the optical filter blocks radiation below 190 nm and above 230 nm,
wherein the UV-C light source and optical filter are disposed within a bezel of the cylindrical housing,
wherein the UV-C light source is a KrCl excimer lamp,
wherein the at least one target location to be disinfected is at least one of a surface location and an airborne location,
wherein the optimal placement is based on at least one of room size and a selected target microorganism,
wherein room size equates to square footage when the target location is a surface location,
wherein room size equates to cubic dimension when the target location is an airborne location;
adjusting a target dosage of the disinfection cycle,
wherein the target dosage is based on a cubic dimension of the enclosed space;
receiving a manual disinfection cycle input by an operator of the disinfection system,
wherein the disinfection cycle is a continuous cycle,
wherein a continuous cycle is in increments of 8 hours;
running a disinfection cycle within enclosed space;
increasing a disinfection cycle duration based on a malfunctioning of one or more UV-C light source of a plurality of UV-C light sources;
ceasing the disinfection cycle upon motion detection within the enclosed space;
resuming the disinfection cycle at the point of cessation when motion is no longer detected for a predetermined amount of time;
monitoring a functionality and a total runtime of the UV-C light source;
broadcasting the functionality based on a color coding of an indicator lamp; and
shutting down the disinfection cycle upon completion a duration of the disinfection cycle or a match of an actual dosage with the target dosage.
